(19) United States
(12) Patent Application Publication
(10) Pub. No.: US 2026/0354863 A1
(43) Pub. Date: Nov. 12, 2026

(54) INSERTION TOOL FOR PIERCING SKIN AND PERFORMING BLUNT DISSECTION OF SUBCUTANEOUS TISSUE

(71) Applicant: Medtronic, Inc., Minneapolis, MN (US)

(72) Inventors: Andrew J. Ries, Lino Lakes, MN (US); Kris A. Peterson, Edina, MN (US); Todd M. Zielinski, McGrath, MN (US)

(21) Appl. No.: 19/125,078

(22) PCT Filed: Oct. 5, 2023
(86) PCT No.: PCT/US2023/076045

§ 371 Date: Apr. 28, 2025

Related U.S. Application Data
(60) Provisional application No. 63/381,389, filed on Oct. 28, 2022.

(51) Int. Cl. A61B 17/34; A61B 5/00; A61B 5/29

(52) U.S. Cl. CPC A61B 17/3468; A61B 5/29; A61B 5/686; A61B 2017/3456; A61B 2560/063

(57) ABSTRACT

This disclosure is directed to devices, systems, and techniques for inserting medical devices. An insertion tool is configured to insert a medical device in subcutaneous tissue of a patient. The insertion tool comprises a sharp member configured to pierce the subcutaneous tissue of the patient to create an opening and a blunt member configured to form a pocket in the subcutaneous tissue via blunt dissection by advancing into the opening. The sharp member may be configured to move along a longitudinal axis of the insertion tool relative to the blunt member. The insertion tool also includes a handle that forms a channel, wherein the handle is configured to receive the medical device in the channel; and a plunger configured to move in the channel, wherein the plunger is configured to push the medical device into the pocket as the plunger distally advances along the channel.

### TECHNICAL FIELD

This application claims the benefit of U.S. Provisional Patent Application No. 63/381,389, filed on Oct. 28, 2022, the entire content of which is incorporated herein by reference.

### TECHNICAL FIELD

The disclosure relates generally to medical device systems and, more particularly, systems for inserting medical devices.

### BACKGROUND

Some types of medical device systems may be configured to monitor one or more physiological parameters of a patient. Such medical device systems may include one or more sensors that detect signals associated with physiological parameters of a patient. These medical devices may be implanted subcutaneously. Subcutaneous medical devices may allow clinicians to obtain patient data without the patient being connected to an external machine and/or present in a clinic. An implantable device that is configured to continuously record one or more physiological parameters allows clinicians to review data over a longer period of time as compared with systems that use external monitoring equipment in a simulated testing situation.

### SUMMARY

In general, the disclosure is directed to devices, systems, and techniques for inserting medical devices using an insertion tool. Insertable cardiac monitors (ICMs) are devices that record one or more patient parameters using electrodes and other sensors. Clinicians use ICMs to record long segments of patient parameters in order to diagnose one or more patient conditions. Some patient conditions only reveal themselves infrequently, so it is beneficial to record long segments of patient data as opposed to only recording short segments when the patient is at a clinic. An insertion tool that is configured to insert an ICM underneath a patient's skin without the need for invasive surgery may provide clinicians the benefit of long, continuously recorded segments of patient data without imposing the inconvenience of major surgery on the patient.

Inserting an ICM may include piercing the patient's skin, creating an opening underneath the skin, and placing the ICM within the opening so that the ICM is configured to record patient data. An example insertion tool may include a sharp member for piercing the subcutaneous tissue, and a blunt member for forming a pocket, e.g., tunnel, in the tissue via blunt dissection. Tools that are configured to both pierce and perform blunt dissection may insert ICMs more effectively as compared with systems that include separate tools for piercing and blunt dissection. The sharp member may include, for example, a flat-edge blade comprising a pointed end. In other examples, the sharp member may include a needle. Needles and small blades are widely used by doctors, nurses, nurse's assistants, pharmacists, and other clinicians and caregivers. This means that an insertion tool having both a sharp member and a blunt member may provide clinicians and caregivers with an easy, convenient, and non-invasive way to insert ICMs underneath the skin that provides data and minimizes patient discomfort.

The sharp member may create an opening in the patient's skin, and the blunt member may enter the opening and create a pocket in the subcutaneous tissue via blunt dissection. The tool may include a plunger configured to push the ICM into the pocket created by the blunt member. Blunt dissection is the careful separation of tissue without “piercing” or “cutting” the tissue using a blade. This means that the blunt member may include an edge that is not sharp, and is configured to separate tissue without cutting the tissue. The sharp member, on the other hand, may perform sharp dissection by cutting into the tissue. This means that it may be beneficial to disengage the sharp member when the blunt member performs blunt dissection, and to engage the sharp member when the sharp member pierces the skin to create the opening.

The techniques of this disclosure may provide one or more advantages. For example, the insertion tool may include a sharp member a blunt member, a handle, and a plunger, giving the caregiver a single tool that pierces the skin, creates an opening, and pushes the ICM into the opening. The insertion tool also has the ability to engage the sharp member when piercing is necessary, and the ability to disengage the sharp member when blunt dissection is necessary. For example, the sharp member may extend so that a distal end of the sharp member is distal to the distal end of the blunt member, and the sharp member may withdraw so that the distal end of the sharp member is proximal to the blunt distal end of the blunt member. This means that the distal end of the insertion tool may toggle between being sharp and being blunt.

Tools that are capable of both piercing and blunt dissection may provide caregivers with a more seamless and less invasive method of inserting ICMs as compared with systems including separate tools for piercing and blunt dissection. For example, some medical devices are implanted via invasive surgical procedures that require the patient to be admitted to a hospital, undergo general anesthesia, and remain in the hospital to recover. Other medical procedures are less invasive than major surgery, and can be performed outside of a hospital and with no anesthesia or local anesthesia that does not render the patient unconscious. Examples of minor medical procedures that can be performed outside of a hospital may involve injecting a patient with one or more substances (e.g., vaccines) and drawing blood samples. An example insertion tool may, in some cases, insert a medical underneath the patient's skin without performing a more invasive surgical procedure. Insertion tools that are configured for blunt dissection and sharp dissection may be better suited for a less invasive insertion of a medical device as compared with systems that use separate tools for sharp dissection and blunt dissection.

In some examples, an insertion tool is configured to insert a medical device in subcutaneous tissue of a patient. The insertion tool comprises a sharp member configured to pierce the subcutaneous tissue of the patient to create an opening and a blunt member configured to form a pocket in the subcutaneous tissue via blunt dissection by advancing into the opening. The sharp member may be configured to move along a longitudinal axis of the insertion tool relative to the blunt member. The insertion tool also includes a handle that forms a channel, wherein the handle is configured to receive the medical device in the channel; and a plunger configured to move in the channel, wherein the plunger is configured to push the medical device into the pocket as the plunger distally advances along the channel.

In some examples, a medical device system includes a medical device; and an insertion tool configured to insert the medical device in subcutaneous tissue of a patient. The insertion tool comprises a sharp member configured to pierce the subcutaneous tissue of the patient to create an opening and a blunt member configured to form a pocket in the subcutaneous tissue via blunt dissection by advancing into the opening. The sharp member may be configured to move along a longitudinal axis of the insertion tool relative to the blunt member. The insertion tool also includes a handle that forms a channel, wherein the handle is configured to receive the medical device in the channel; and a plunger configured to move in the channel, wherein the plunger is configured to push the medical device into the pocket as the plunger distally advances along the channel.

In some examples, a method includes piercing, using a sharp member of an insertion tool configured to insert a medical device in subcutaneous tissue of a patient, the subcutaneous tissue of the patient to create an opening; and forming, using a blunt member of the insertion tool, a pocket in the subcutaneous tissue via blunt dissection by advancing into the opening, wherein the sharp member is configured to move along a longitudinal axis of the insertion tool relative to the blunt member. Additionally, the method includes receiving, by a handle of the insertion tool that forms a channel, the medical device in the channel; and pushing, by a plunger of the insertion tool configured to move in the channel, the medical device into the pocket as the plunger distally advances along the channel.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIGS. 1A and 1B are conceptual diagrams illustrating a first side of an insertion tool for inserting an implantable medical device (IMD) within subcutaneous tissue of a patient, in accordance with one or more techniques of this disclosure.

FIGS. 2A and 2B are conceptual diagrams illustrating a second side of an insertion tool for inserting the IMD within subcutaneous tissue of a patient, in accordance with one or more techniques of this disclosure.

FIGS. 3A-3C are conceptual diagrams illustrating insertion tools that include sharp members of different widths, in accordance with one or more techniques of this disclosure.

FIG. 4 is a conceptual drawing illustrating an example configuration of an IMD configured to be inserted under a patient's skin using an insertion tool, in accordance with one or more techniques described herein.

FIG. 5 is a conceptual diagram illustrating a perspective view of an insertion tool for inserting an IMD in a patient while the insertion tool occupies a first configuration, in accordance with one or more techniques described herein.

FIG. 6 is a conceptual diagram illustrating a perspective view of an insertion tool for inserting an IMD in a patient while the insertion tool occupies a second configuration, in accordance with one or more techniques described herein.

FIG. 7 is a conceptual diagram illustrating a perspective view of an insertion tool including an IMD within a channel formed by the insertion tool, in accordance with one or more techniques described herein.

FIG. 8 is a conceptual diagram illustrating a perspective view of a distal portion of an insertion tool, in accordance with one or more techniques described herein.

FIG. 9 is a flow diagram illustrating an example method for using an insertion tool to insert an IMD, in accordance with one or more techniques of this disclosure.

### DETAILED DESCRIPTION

This disclosure describes techniques for placing a medical device under the skin of a patient using an insertion tool. In some examples, subcutaneous medical devices may record one or more biometric signals of a patient, where the biometric signals indicate patient parameters. It may be beneficial to place a medical device under the patient's skin using a minimally invasive insertion procedure so that the medical device can collect long segments of data, and the patient does not suffer the consequences of an invasive surgery. Techniques for placing a medical device under the skin of the patient may include puncturing the patient's skin using a sharp blade or a sharp needle to create an opening, forming a pocket in subcutaneous tissue via blunt dissection, and pushing the medical device into the opening.

An example insertion tool may include a sharp member for piercing the skin, a blunt member for creating a pocket in subcutaneous tissue via blunt dissection, a handle that forms a channel for receiving a medical device, and a plunger configured to push the medical device into the pocket. In this way, the insertion tool may be capable of blunt dissection and sharp dissection. The tool may use sharp dissection to pierce the skin and blunt dissection to create the pocket within subcutaneous tissue. To ensure that a sharp point or a sharp edge of the sharp member does not prevent the insertion tool from performing blunt dissection, the sharp member may be configured to move along a longitudinal axis of the insertion tool relative to the blunt member. In other words, the sharp member may advance beyond a distal end of the blunt member when the tool is used to pierce the skin, and the sharp member may retreat to a location proximal to the distal end of the blunt member when the tool is used for blunt dissection.

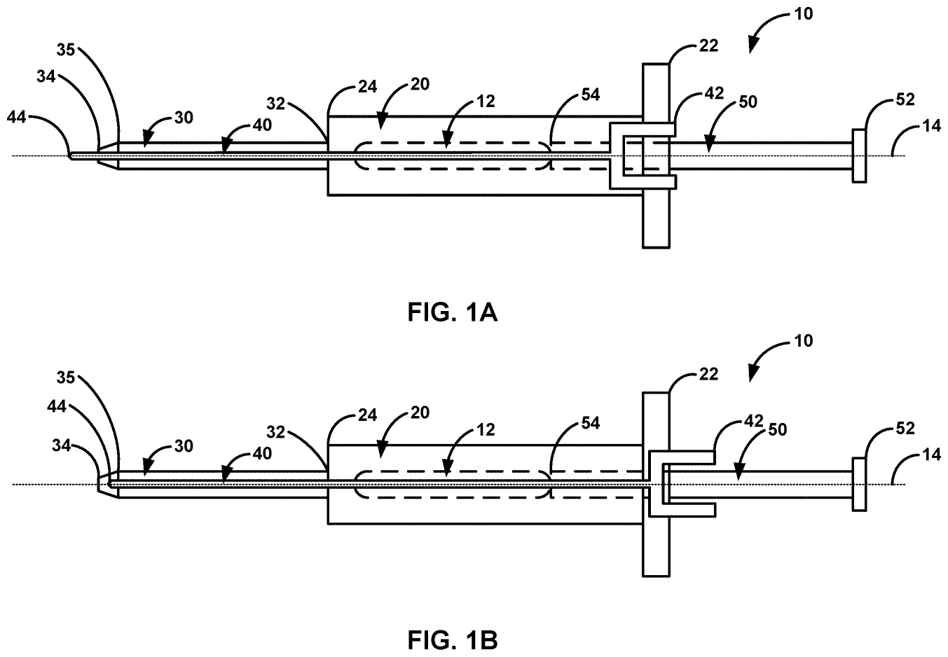

FIGS. 1A and 1B are conceptual diagrams illustrating a first side of an insertion tool 10 for inserting an implantable medical device (IMD) 12 within subcutaneous tissue of a patient, in accordance with one or more techniques of this disclosure. FIG. 1A is a conceptual diagram illustrating the insertion tool 10 in a first arrangement, and FIG. 1B is a conceptual diagram illustrating the insertion tool 10 in a second arrangement. Insertion tool 10 extends along a longitudinal axis 14. As seen in FIGS. 1A and 1B, insertion tool 10 includes a handle 20, a blunt member 30, a sharp member 40, and a plunger 50. Handle 20 includes a proximal end 22 and a distal end 24, blunt member 30 includes a proximal end 32 and a distal end 34, sharp member 40 includes a proximal end 42 and a distal end 44, and plunger 50 includes a proximal end 52 and a distal end 54. In some examples, FIGS. 1A and 1B show a first side of insertion tool 10. A view from a second side of insertion tool 10, opposite the first side, may provide a different perspective of insertion tool 10.

As applied herein, the terms “implantable medical device” and “IMD” may refer to any medical device configured to be placed underneath the patient's skin. One example of an IMD is an insertable cardiac monitor (ICM), which is inserted underneath the skin using minimally invasive procedures. Other examples of IMDs include pacemakers and implantable cardioverter defibrillators (ICDs), which require more invasive surgical procedures to implant. In other words, the terms “implantable medical device” and “IMD” may refer to any device configured to be placed underneath the skin, regardless of the invasiveness of the procedures used to place the IMD underneath the skin.

Insertion tool 10 may represent a single tool that is configured to create a pocket in the subcutaneous tissue of a patient, and place IMD 12 within the pocket. For example, sharp member 40 may pierce the patient's skin to create an opening, blunt member 30 may enter the opening in the patient's skin and create a pocket in the patient's subcutaneous tissue via blunt dissection. In some examples, blunt member 30 may dilate the opening created by the sharp member 40. For example, a width of sharp member 40 may be less than a width of blunt member 30 and when blunt member 30 enters the opening created by sharp member 40, blunt member 30 may dilate the opening. Plunger 50 may push IMD 12 out of a channel in handle 20, onto a surface of blunt member 30 and into the pocket in the patient's subcutaneous tissue. In some examples, a width of IMD 12 may be greater than a width of blunt member 30, and IMD 12 may further dilate the opening originally created by sharp member 40 and subsequently dilated by blunt member 30. Blunt member 30 may withdraw from the pocket in the patient's subcutaneous tissue, leaving IMD 12 in the pocket. Single insertion tools such as insertion tool 10 that are configured for piercing the skin, creating a pocket, and inserting an IMD in the pocket may better enable caregivers to insert IMDs as compared with systems that use separate tools for piercing the skin and performing blunt dissection.

Furthermore, single insertion tools may be better configured for use outside of traditional hospital settings. For example, insertion tool 10 may be better configured for use by non-surgeon caregivers such as nurses, nursing assistants, and pharmacists as compared with systems that use a separate “incision tool” for piercing the skin. For example, sharp member 40 of insertion tool 10 may pierce the patient's skin, obviating a need to create a surgical incision using a separate incision tool. Blunt member 30 may create a pocket underneath the patient's skin and plunger 50 may insert IMD 12 into the pocket. In other words, the single insertion tool 10 may provide techniques for inserting IMD 12 that are more like an injection than a major surgical procedure. In some examples, IMD 12 may comprise an Insertable Cardiac Monitor (ICM).

Handle 20 may, in some examples, extend from the proximal end 22 of the handle 20 to the distal end 24 of the handle 20. In some examples, handle 20 may form a channel (not illustrated in FIGS. 1A and 1B) that is configured to receive IMD 12. Although the channel formed by handle 20 might not be visible in FIGS. 1A and 1B, the channel may be visible in other views of insertion tool 10 from other perspectives. IMD 12 is shown as a dotted line in FIGS. 1A and 1B, because IMD 12 may not be visible from the perspective of FIGS. 1A and 1B, but the dotted line may indicate an example position of IMD 12 relative to handle 20.

In some examples, blunt member 30 may extend from a proximal end 32 to a distal end 34. Blunt member 30 distally from the distal end 24 of the handle 20. For example, blunt member 30 may extend distally from the distal end 24 of handle 20 so that the proximal end 32 of blunt member 30 is connected to the distal end 24 of handle 20. In some examples, blunt member 30 is formed integrally with handle 20 such that proximal end 32 of blunt member 30 is integral with the distal end 24 of handle 20. A width of blunt member 30 at the proximal end 32 of blunt member 30 may, in some examples, be approximately the same as a width of a central portion of IMD 12, but this is required. A width of blunt member 30 at the proximal end 32 of blunt member 30 may be less than the width of the central portion of IMD 12. When the IMD 12 exits the channel formed by handle 20 and enters the pocket created by blunt member 30, the IMD 12 may enlarge the pocket and fit snugly within the pocket.

A distal portion of blunt member 30 may be tapered so that a width of the distal end 34 is less than a width of a central portion of blunt member 30. The distal portion of blunt member 30 may include the portion of blunt member 30 between distal end 34 and point 35. As seen in FIGS. 1A and 1B, the width of blunt member 30 tapers from point 35 to distal end 34. In some examples, distal end 34 may include a flat edge, and the distal portion of blunt member 30 might not include sharp edges. This allows blunt member 30 to perform blunt dissection without cutting tissue with sharp edges. Although FIGS. 1A and 1B show blunt member 30 as having a tapered distal portion with a flat end, this is not the only configuration for blunt member 30. In some examples, the distal end of blunt member 30 may include a rounded distal end or a spheroid distal end. In any case, when the distal end of blunt member 30 advances into tissue of a patient, blunt member 30 may perform blunt dissection without cutting the tissue using sharp edges. In some examples, the distal end of blunt member 30 does not taper to a single point comprising an acute angle. In some examples, the distal end of blunt member 30 may include two or more obtuse angles, wherein each obtuse angle of the two or more obtuse angles is greater than 90 degrees. In some examples, the distal end of blunt member 30 may be rounded such that the distal end of blunt member 30 does not include angles.

Sharp member 40 may extend from proximal end 42 to distal end 44. Sharp member 40 may extend along the longitudinal axis 14 parallel to handle 20, blunt member 30, and plunger 50. In some examples, sharp member 40 may be configured to move along longitudinal axis 14 relative to handle 20 and blunt member 30. The sharp member 40 as seen in FIG. 1A occupies a first position relative to handle 20 and blunt member 30 and the sharp member 40 as seen in FIG. 1A occupies a second position relative to blunt member 30. For example, as seen in FIG. 1A, the distal end 44 of sharp member 40 extends distally beyond the distal end 34 of blunt member 30, and the proximal end 42 of sharp member 40 is close to the proximal end 22 of handle 20. As seen in FIG. 1B, the distal end 44 of sharp member 40 is proximal to the distal end 34 of blunt member 30, and the proximal end 42 of sharp member 40 extends proximally from proximal end 22 of handle 20. In some examples, the distal end 44 of sharp member 40 may include a pointed distal end. For example, sharp member 40 may have a substantially consistent width extending along most of a length of sharp member 40. At distal end 44, the width of sharp member 40 may taper to a point. In some examples, the point may represent an acute angle that is less than 60 degrees. In some examples, the point may represent an acute angle that is less than 20 degrees. In some examples, the point may represent an acute angle that is less than 10 degrees.

In some examples, when the sharp member 40 pierces the subcutaneous tissue of the patient, the IMD 12 is outward of the sharp member and the blunt member relative to the patient's body. When blunt member 30 advances into subcutaneous tissue to form a pocket, IMD 12 may, in some cases, be outward of the blunt member 30 relative to the patient's body. When a portion of the blunt member 30 is within the subcutaneous tissue of the patient, insertion tool 10 may rotate such that IMD 12 is inward of blunt member 30 relative to the patient's body.

Blunt member 30 and sharp member 40 are not limited to having widths as shown in FIGS. 1A and 1B. In some examples, a width of sharp member 40 is within a range from 90% of a width of the blunt member 30. In some examples, a width of sharp member 40 is less than 90% of a width of the blunt member 30. In some examples, a width of sharp member 40 is less than 50% of a width of the blunt member 30. In some examples, a width of sharp member 40 is less than 30% of a width of the blunt member 30. In some examples, a width of sharp member 40 is less than 10% of a width of the blunt member 30. In some examples, a width of the sharp member 40 is less than a width of the blunt member 30, and the width of blunt member 30 is less than a width of IMD 12.

To transition from the first position of FIG. 1A to the second position of FIG. 1B, sharp member 40 may move proximally along longitudinal axis 14 relative to blunt member 30 and handle 20 so that distal end 44 retreats to a position proximal to the distal end 34 of blunt member 30. To transition from the second position of FIG. 1B to the first position of FIG. 1A, sharp member 40 may move distally along longitudinal axis 14 relative to handle 20 and blunt member 30 so that distal end 44 of sharp member 40 extends beyond the distal end 34 of blunt member 30.

In some examples, one or more springs (not illustrated in FIGS. 1A and 1B) may be connected to handle 20 and sharp member 40. A user control system (not illustrated in FIGS. 1A and 1B) may allow a user to control whether sharp member 40 occupies the first position of FIG. 1A or the second position of FIG. 1B. For example, the user control system may secure the sharp member 40 in the first position where proximal end 42 of the sharp member 40 is secured to the proximal end 22 of handle 20, resisting a force applied by the one or more springs. When the user control system releases the sharp member 40 so that the proximal end 42 of sharp member 40 is no longer secured to the proximal end 22 of handle 20, the one or more springs may push sharp member 40 proximally along longitudinal axis 14 so that sharp member 40 occupies the second position of FIG. 1B.

Plunger 50 may extend along the longitudinal axis 14 from a proximal end 52 to a distal end 54. From the view of insertion tool 10 as illustrated in FIGS. 1A and 1B, only a proximal portion of plunger 50 may be visible. A distal portion of plunger 50 may be configured to be located within a channel formed by handle 20. The distal portion of plunger 50 within the channel is shown in dotted lines on FIGS. 1A and 1B. As seen in FIGS. 1A and 1B, the distal end 54 of plunger 50 is configured to make contact with a proximal end of IMD 12 within the channel formed by handle 20. Plunger 50 may be configured to move along longitudinal axis 14 relative to handle 20. As plunger advances distally along longitudinal axis 14, plunger 50 may push IMD 12 out of a distal opening of the channel formed by handle 20. In some examples, plunger 50 pushes IMD 12 out of the distal opening of the channel formed by handle 20 when a portion of the blunt member is located within the pocket formed in the patient's skin and when the handle 20 is outside of the pocket. In some examples, plunger 50 pushes IMD 12 out of the distal opening of the channel formed by handle 20 when insertion tool 10 is rotated such that IMD 12 is inward of the blunt member 30 relative to the patient's body.

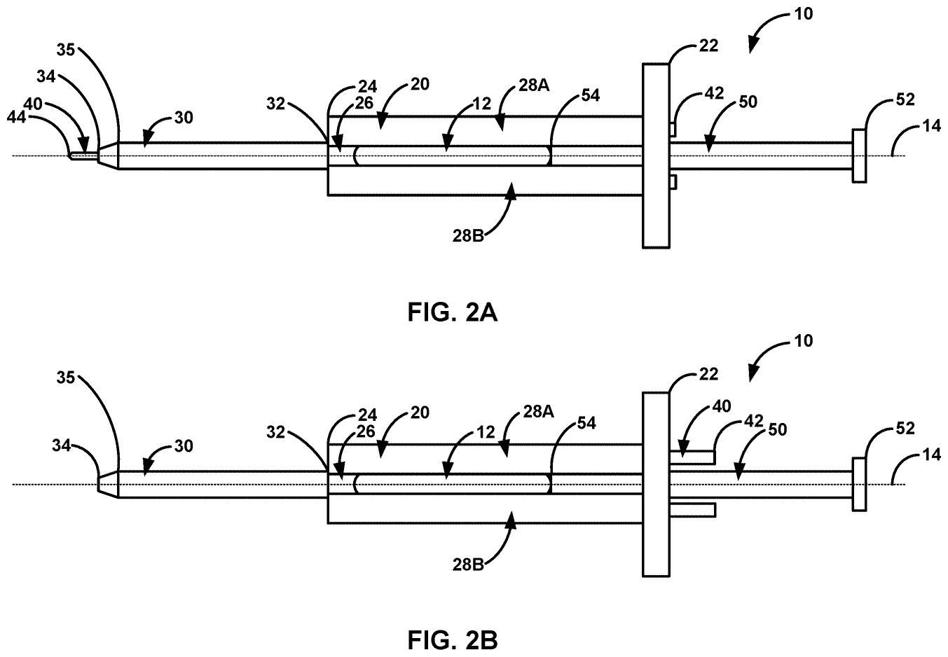

FIGS. 2A and 2B are conceptual diagrams illustrating a second side of an insertion tool 10 for inserting IMD 12 within subcutaneous tissue of a patient, in accordance with one or more techniques of this disclosure. FIG. 2A is a conceptual diagram illustrating the second side of insertion tool 10 in the first arrangement also shown in FIG. 1A, and FIG. 2B is a conceptual diagram illustrating the second side of insertion tool 10 in the second arrangement also shown in FIG. 1B. Insertion tool 10 extends along a longitudinal axis 14. In some examples, FIGS. 1A and 1B show a first side of insertion tool 10, and FIGS. 2A and 2B show a second side of insertion tool 10 that is opposite the first side. As seen in FIGS. 2A and 2B, insertion tool 10 includes handle 20, blunt member 30, sharp member 40, and plunger 50.

Handle 20 includes a bottom wall portion 26 and side wall portions 28A and 28B (collectively, “side wall portions 28”). Bottom wall portion 26 and side wall portions 28 may form a channel that is configured to receive IMD 12. For example, side wall portions 28 may extend upwards from bottom wall portion 26 from the perspective of FIGS. 2A and 2B. This means that bottom wall portion 26 and side wall portions 28 may represent three walls of an open channel that is configured to receive IMD 12. In some examples, the channel is configured to receive IMD 12 so that there is space between IMD 12 and each of side wall portions 28. In some examples, the channel is configured to receive IMD 12 so that the sides of IMD 12 are flush to side wall portions 28. The configuration of handle 20 shown in FIGS. 2A and 2B is not the only possible configuration of handle 20. In some cases, IMD 12 may include one or more top wall portions that obscure at least a portion of the top surface of IMD 12.

In some examples, a cross-section of the channel formed by handle 20 may be rectangular in shape, but this is not required. In some examples, a cross-section of the channel formed by handle 20 may include rounded corners to match rounded corners of IMD 12 and/or rounded corners of plunger 50. The cross-section of the channel formed by handle 20 may also have a shape other than a rectangle or a rectangular shape with rounded corners. The cross-section of the channel may have any shape representing any combination of straight lines and/or curved lines. In some examples, a top of handle 20 rises above a top of IMD 12 such that a cross-section of IMD 12 is completely within a cross-section of handle 20.

Blunt member 30 may extend distally from the distal end 24 of handle 20. In some examples, Blunt member 30 may extend from a bottom portion of the distal end 24 of handle 20 from the perspective of FIGS. 2A and 2B. For example, bottom wall portion 26 may be located at a bottom of insertion tool 10 from the perspective of FIGS. 2A and 2B, and Blunt member 30 may extend distally from a distal end of the bottom wall portion 26. Side wall portions 28 may extend upwards from the bottom wall portion 26 and above the blunt member 30 from the perspective of FIGS. 2A and 2B.

Sharp member 40 may be configured to be located on the back of insertion tool 10 from the perspective of FIGS. 2A and 2B, and located on the front of insertion tool 10 from the perspective of FIGS. 1A and 1B. Since sharp member 40 is located on the back of insertion tool 10 from the perspective of FIGS. 2A and 2B, sharp member 40 is mostly obscured by the blunt member 30 and the handle 20 in FIGS. 2A and 2B. When sharp member 40 extends beyond the distal end blunt member 30 as shown by FIG. 2A, a distal portion of sharp member 40 including is visible from the perspective of FIG. 2A. When sharp member 40 retreats proximally along longitudinal axis 14 so that the distal end 44 of sharp member 40 is proximal to the distal end 34 of blunt member as shown in FIG. 2B, the distal portion of sharp member 40 may be obscured by blunt member 30 from the perspective of FIG. 2B. But in the second position of sharp member 40 shown in FIG. 2B, a proximal portion of sharp member 40 may extend proximally from the proximal end 22 of handle 20.

Plunger 50 may be configured to move along longitudinal axis 14 and within the channel formed by handle 20. In some examples, plunger 50 may be configured to move proximally within the channel formed by handle 20, pushing IMD 12 out of a distal opening of the channel and onto a surface of blunt member 30. In some examples, plunger 50 may push IMD 12 onto the surface of blunt member 30 that is shown in FIGS. 2A and 2B. Sharp member 40 may be configured to be located on a surface of blunt member 30 that is opposite the surface of blunt member 30 that is shown in FIGS. 2A and 2B. This means that sharp member 40 and IMD 12 are both configured to move along longitudinal axis 14 relative to blunt member 30, on opposite sides of blunt member 30.

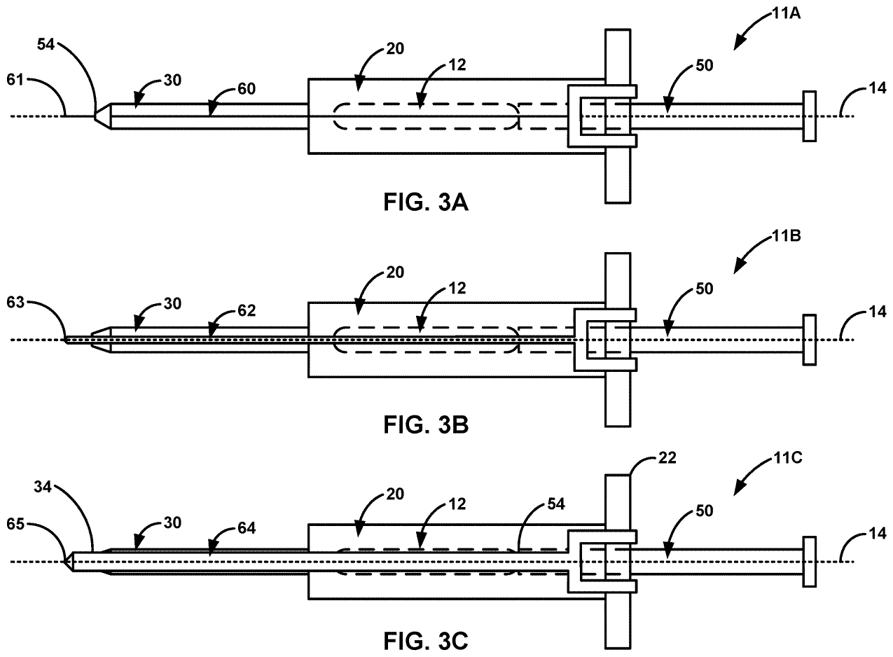

FIGS. 3A-3C are conceptual diagrams illustrating insertion tools 11A-11C that include sharp members of different widths, in accordance with one or more techniques of this disclosure. Insertion tool 11A of FIG. 3A includes a first sharp member 60 having a first width, insertion tool 11B of FIG. 3B includes a second sharp member 62 having a second width, and insertion tool 11C of FIG. 3C includes a third sharp member 64 having a third width. In some examples, insertion tool 11B may be an example of insertion tool 10 of FIGS. 1-2. But example insertion tools described herein are not limited to having sharp members of the configuration shown in FIGS. 1-2.

As seen in FIG. 3A, the first sharp member 60 may represent a needle having a sharp end 61. In some examples, sharp end 61 may include a sharp point configured to pierce the skin of a patient to create an opening. A distal portion of blunt member 30 may advance into the opening and create a pocket in subcutaneous tissue of the patient via blunt dissection. In some examples, it may be beneficial for the first sharp member 60 to include a needle having a smaller width as compared with second sharp member 62 and third sharp member 64. Needles are widely used by caregivers in hospital and non-hospital settings. This means that many caregivers may be able to use insertion tool 11A in a range of clinical settings.

Second sharp member 62 of insertion tool 11B may have a sharp end 63 that is configured to pierce the skin of a patient to create an opening. In some examples, the sharp end 63 of sharp member 62 may comprise a tapered distal portion of second sharp member 62 having a sharp edge and/or a sharp point. As seen in FIG. 3B, the second sharp member 62 may have a width that is greater than a width of the first sharp member 60. Since a width of second sharp member 62 is greater than a width of first sharp member 60, second sharp member 62 may be configured to create a larger opening in the patient's skin as compared with the first sharp member 60.

Third sharp member 64 of insertion tool 11C may have a sharp end 65 that is configured to pierce the skin of a patient to create an opening. In some examples, the sharp end 65 of sharp member 64 may comprise a tapered distal portion of third sharp member 64 having a sharp edge and/or a sharp point. As seen in FIG. 3B, the third sharp member 64 may have a width that is greater than a width of the second sharp member 62. Since a width of third sharp member 64 is greater than a width of second sharp member 62, third sharp member 64 may be configured to create a larger opening in the patient's skin as compared with the second sharp member 62.

In some examples, a configuration of the blunt member 30 may depend on a width of the respective sharp member. That is, for sharp members such as the first sharp member 60 that have small widths, the distal end of the blunt member 30 may need to taper to a small width so that the blunt member 30 can enter the opening created by the sharp member and create a pocket in subcutaneous tissue via blunt dissection. But when the sharp member has a larger width, it might not be necessary for the distal end of the blunt member 30 to taper to a small width. Sharp members 60, 62, and 64 are not the only example configurations for sharp members. Sharp members of other configurations may be configured to perform one or more techniques of this disclosure.

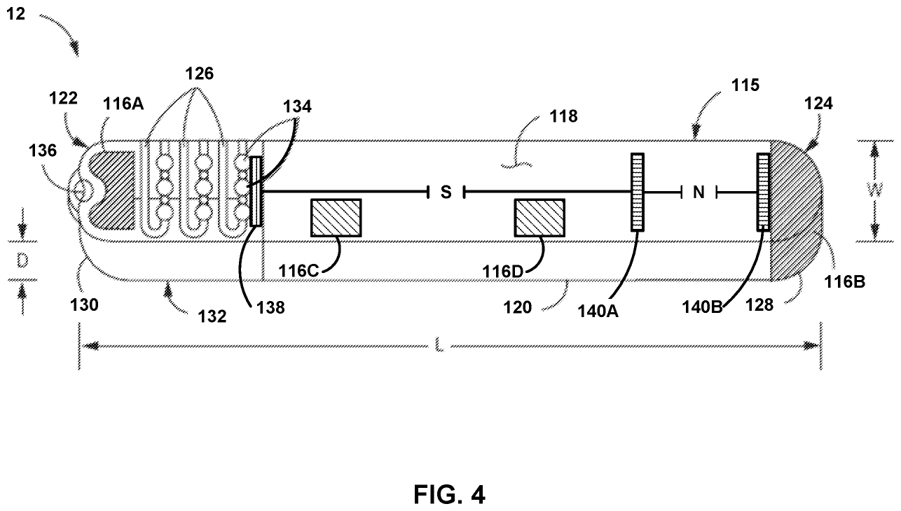

FIG. 4 is a conceptual drawing illustrating an example configuration of an IMD 12 configured to be inserted under a patient's skin using insertion tool 10, in accordance with one or more techniques described herein. In the example shown in FIG. 4, IMD 12 may represent a leadless, subcutaneously-insertable monitoring device including a housing 115, proximal electrode 116A, and distal electrode 116B. Housing 115 may further include first major surface 118, second major surface 120, proximal end 122, and distal end 124. In some examples, IMD 12 may include one or more additional electrodes 116C, 116D positioned on one or both of major surfaces 118, 120 of IMD 12. Housing 115 encloses electronic circuitry located inside the IMD 12, and protects the circuitry contained therein from fluids such as body fluids. In some examples, electrical feedthroughs provide electrical connection of electrodes 116A-116D, and antenna 126, to circuitry within housing 115. In some examples, electrode 116B may be formed from an uninsulated portion of conductive housing 115. The techniques described herein are not limited to the IMD 12 having the configuration shown in FIG. 4. Insertion tool 10 may place IMDs having configurations other than the configuration shown in FIG. 4 within subcutaneous tissue of a patient.

In the example shown in FIG. 4, IMD 12 is defined by a length L, a width W, and thickness or depth D. In this example, IMD 12 is in the form of an elongated rectangular prism in which length L is significantly greater than width W, and in which width W is greater than depth D. However, other configurations of IMD 12 are contemplated, such as those in which the relative proportions of length L, width W, and depth D vary from those described and shown in FIG. 4. In some examples, the geometry of the IMD 12, such as the width W being greater than the depth D, may be selected to allow IMD 12 to be inserted under the skin of the patient by an insertion tool using a minimally invasive procedure and to remain in the desired orientation during insertion. In addition, IMD 12 may include radial asymmetries (e.g., the rectangular shape) along a longitudinal axis of IMD 12, which may help maintain the device in a desired orientation following insertion.

In some examples, a distance between proximal electrode 116A and distal electrode 116B may be within a range from 3 millimeters (mm) to 55 mm, within a range from 3 mm to 20 mm, or within a range from 20 mm to 55 mm. Overall, IMD 12 may have a length L within a range from 5 mm to 60 mm, within a range from 5 mm to 10 mm, within a range from 5 mm to 20 mm, within a range from 20 mm to 60 mm, or within a range from 40 mm to 60 mm. In some examples, the width W of major surface 118 may be within a range from 3 mm to 20 mm, within a range from 3 mm to 10 mm, or within a range from 5 mm to 15 mm. In some examples, a depth D of IMD 12 may be within a range from 2 mm to 15 mm, within a range from 2 mm to 9 mm, or within a range from 5 mm to 15 mm. In other examples, the depth D of IMD 12 may be within a range from 2 mm to 5 mm. In any such examples, IMD 12 is sufficiently compact to be inserted within the subcutaneous space of the patient in the region of a pectoral muscle.

IMD 12, according to an example of the present disclosure, may have a geometry and size designed for ease of insertion and patient comfort. Examples of IMD 12 described in this disclosure may have a volume of 3 cubic centimeters (cm³) or less, 1.5 cm³ or less, or any volume therebetween. In addition, in the example shown in FIG. 4, proximal end 122 and distal end 124 may be rounded to reduce discomfort and irritation to surrounding tissue once inserted under the skin of the patient.

In the example shown in FIG. 4, first major surface 118 of IMD 12 faces outward towards the skin, when IMD 12 is inserted within the patient, whereas second major surface 120 faces inward toward musculature of the patient. Thus, first and second major surfaces 118, 120 may face in directions along a sagittal axis of the patient, and this orientation may be maintained upon insertion.

Proximal electrode 116A and distal electrode 116B may be used to sense cardiac electrograms (EGMs) (e.g., cardiac electrocardiograms (ECGs)) when IMD 12 is inserted subcutaneously in the patient. In some examples, processing circuitry of IMD 12 also may determine whether cardiac EGMs of the patient are indicative of arrhythmia or other abnormalities, which processing circuitry of IMD 12 may evaluate in determining whether a medical condition (e.g., heart failure, sleep apnea, or chronic obstructive pulmonary disease (COPD)) of the patient has changed. The cardiac EGMs may be stored in a memory of the IMD 12. In some examples, data derived from the EGMs may be transmitted via antenna 126 to another medical device, such as an external device. In some examples, one or both of electrodes 116A and 116B also may be used by IMD 12 to collect one or more impedance signals (e.g., a subcutaneous tissue impedance) during impedance measurements performed by IMD 12. In some examples, such impedance values detected by IMD 12 may reflect a resistance value associated with a contact between electrodes 116A, 116B, and target tissue of the patient. Additionally, in some examples, electrodes 116A, 116B may be used by communication circuitry of IMD 12 for tissue conductance communication (TCC) communication with an external device or another device.

In the example shown in FIG. 4, proximal electrode 116A is in close proximity to proximal end 122, and distal electrode 116B is in close proximity to distal end 124 of IMD 12. In this example, distal electrode 116B is not limited to a flattened, outward facing surface, but may extend from first major surface 118, around rounded edges 128 or end surface 130, and onto the second major surface 120 in a three-dimensional curved configuration. As illustrated, proximal electrode 116A is located on first major surface 118 and is substantially flat and outward facing. However, in other examples not shown here, proximal electrode 116A and distal electrode 116B both may be configured like proximal electrode 116A shown in FIG. 4, or both may be configured like distal electrode 116B shown in FIG. 4. In some examples, additional electrodes 116C and 116D may be positioned on one or both of first major surface 118 and second major surface 120, such that a total of four electrodes are included on IMD 12. Any of electrodes 116A-116D may be formed of a biocompatible conductive material. For example, any of electrodes 116A-116D may be formed from any of stainless steel, titanium, platinum, iridium, or alloys thereof. In addition, electrodes of IMD 12 may be coated with a material such as titanium nitride or fractal titanium nitride, although other suitable materials and coatings for such electrodes may be used.

In the example shown in FIG. 4, proximal end 122 of IMD 12 includes header assembly 132 having one or more of proximal electrode 116A, antenna 126, anti-migration projections 134, and suture hole 136. Antenna 126 is located on the same major surface (e.g., first major surface 118) as proximal electrode 116A, and may be an integral part of header assembly 132. In other examples, antenna 126 may be formed on the major surface opposite from proximal electrode 116A, or, in still other examples, may be incorporated within housing 115 of IMD 12. Antenna 126 may be configured to transmit or receive electromagnetic signals for communication. For example, antenna 126 may be configured to transmit to or receive signals from a programmer via inductive coupling, electromagnetic coupling, tissue conductance, Near Field Communication (NFC), Radio Frequency Identification (RFID), Bluetooth®, Wi-Fi®, or other proprietary or non-proprietary wireless telemetry communication schemes. Antenna 126 may be coupled to communication circuitry of IMD 12, which may drive antenna 126 to transmit signals to an external device and may transmit signals received from the external device to processing circuitry of IMD 12 via communication circuitry.

IMD 12 may include several features for retaining IMD 12 in position once subcutaneously inserted in the patient. For example, as shown in FIG. 4, housing 115 may include anti-migration projections 134 positioned adjacent antenna 126. Anti-migration projections 134 may include a plurality of bumps or protrusions extending away from first major surface 118 and may help prevent longitudinal movement of IMD 12 after insertion in the patient. In other examples, anti-migration projections 134 may be located on the opposite major surface as proximal electrode 116A and/or antenna 126. In addition, in the example shown in FIG. 4 header assembly 132 includes suture hole 136, which provides another means of securing IMD 12 to the patient to prevent movement following insertion. In the example shown, suture hole 136 is located adjacent to proximal electrode 116A. In some examples, header assembly 132 may include a molded header assembly made from a polymeric or plastic material, which may be integrated or separable from the main portion of IMD 12.

Electrodes 116A and 116B may be used to sense cardiac EGMs, as described above. Additional electrodes 116C and 116D may be used to sense subcutaneous tissue impedance, in addition to or instead of electrodes 116A, 116B, in some examples. In some examples, processing circuitry of IMD 12 may determine an impedance value of the patient based on signals received from at least two of electrodes 116A-116D. For example, processing circuitry of IMD 12 may generate one of a current or voltage signal, deliver the signal via a selected two or more of electrodes 116A-116D, and measure the resulting other of current or voltage. Processing circuitry of IMD 12 may determine an impedance value based on the delivered current or voltage and the measured voltage or current.

In the example shown in FIG. 4, IMD 12 includes light emitter(s) 138 and a proximal light detector 140A and a distal light detector 140B (collectively, “light detectors 140”) positioned on housing 115 of IMD 12. Light detector 140A may be positioned at a distance S from light emitter(s) 138, and a distal light detector 140B positioned at a distance S+N from light emitter(s) 138. In other examples, IMD 12 may include only one of light detectors 140A, 140B, or may include additional light emitters and/or additional light detectors. Collectively, light emitter(s) 138 and light detectors 140A, 140B may include an optical sensor, which may be used to determine one or more oxygenation values of a patient such as pulse oximetry (SpO₂), tissue oxygen saturation (StO₂), arterial blood oxygen saturation (SaO₂), venous oxygen saturation (SvO₂), or any combination thereof. Light emitter(s) 138 include a light source, such as an LED, that may emit light at one or more wavelengths within the visible (VIS) and/or near-infrared (NIR) spectra. For example, light emitter(s) 138 may emit light at one or more of about 660 nanometer (nm), 720 nm, 760 nm, 800 nm, or at any other suitable wavelengths. Light detectors 140A, 140B may detect light emitted by light emitter(s) 138. Based on one or more parameters of light emitted by light emitter(s) 138 and one or more parameters of light received by light detectors 140A, 140B, processing circuitry of IMD 12 may determine one or more oxygenation values of the patient.

As shown in FIG. 4, light emitter(s) 138 may be positioned on header assembly 132, although, in other examples, one or both of light detectors 140A, 140B may additionally or alternatively be positioned on header assembly 132. In some examples, light emitter(s) 138 may be positioned on a medial section of IMD 12, such as part way between proximal end 122 and distal end 124. Although light emitter(s) 138 and light detectors 140A, 140B are illustrated as being positioned on first major surface 118, light emitter(s) 138 and light detectors 140A, 140B alternatively may be positioned on second major surface 120. In some examples, IMD may be inserted such that light emitter(s) 138 and light detectors 140A, 140B face inward when IMD 12 is inserted, toward the muscle of the patient, which may help minimize interference from background light coming from outside the body of the patient. Light detectors 140A, 140B may include a glass or sapphire window, such as described below with respect to FIG. 4B, or may be positioned beneath a portion of housing 115 of IMD 12 that is made of glass or sapphire, or otherwise transparent or translucent.

Light emitter(s) 138 may emit light into a target site of the patient during a technique for determining an oxygenation value of the patient. The target site may generally include the interstitial space around IMD 12 when IMD 12 is inserted in the patient. Light emitter(s) 138 may emit light directionally in that light emitter(s) 138 may direct the signal to a side of IMD 12, such as when light emitter(s) 138 are disposed on the side of IMD 12 that includes first major surface 118. The target site may include the subcutaneous tissue adjacent IMD 12 within the patient.

In some examples, IMD 12 may include one or more additional sensors, such as one or more accelerometers (not illustrated in FIG. 4). Such accelerometers may be 3D accelerometers configured to generate signals indicative of one or more types of movement of the patient, such as gross body movement (e.g., motion) of the patient, patient posture, movements associated with the beating of the heart, or coughing, rales, or other respiration abnormalities. One or more of the parameters monitored by IMD 12 (e.g., impedance, EGM) may fluctuate in response to changes in one or more such types of movement. For example, changes in parameter values sometimes may be attributable to increased patient motion (e.g., exercise or other physical motion as compared to immobility) or to changes in patient posture, and not necessarily to changes in a medical condition. Thus, in some methods of identifying or tracking a medical condition of the patient, it may be advantageous to account for such fluctuations when determining whether a change in a parameter is indicative of a change in a medical condition.

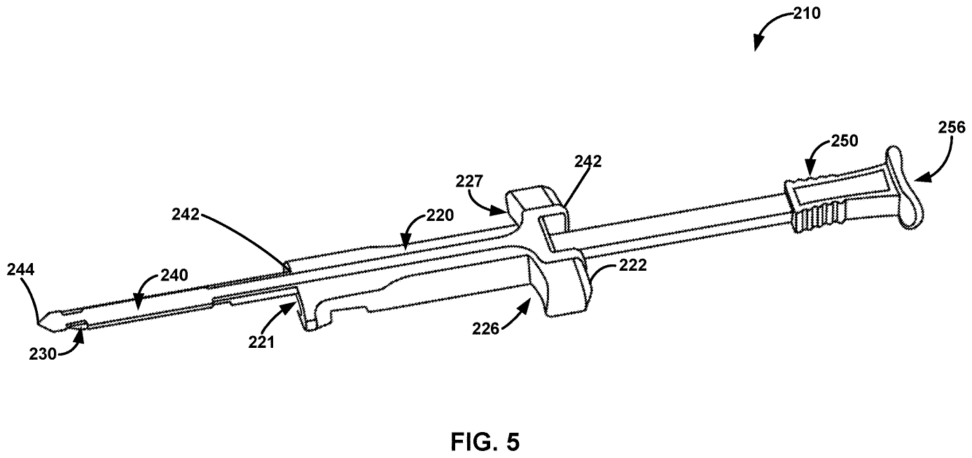

FIG. 5 is a conceptual diagram illustrating a perspective view of an insertion tool 210 for inserting an IMD in a patient while the insertion tool 210 occupies a first configuration, in accordance with one or more techniques described herein. As seen in FIG. 5, insertion tool 210 includes a handle 220, a blunt member 230, a sharp member 240, and a plunger 250. In some examples, handle 220 is an example of handle 20 of FIGS. 1A-2B, blunt member 230 is an example of blunt member 30 of FIGS. 1A-2B, sharp member 240 is an example of sharp member 40 of FIGS. 1A-2B, and plunger 250 is an example of plunger 50 of FIGS. 1A-2B.

The perspective of insertion tool 210 shown in FIG. 5, may show the first side of the insertion tool 210 shown in FIGS. 1A-1B. Although a distal opening 221 of a channel formed by handle 220 is visible in FIG. 5, an IMD received within the channel is not visible because the channel is closed on the first side of the insertion tool 210. The IMD may be visible on a second side of insertion tool 210 that includes an opening. Insertion tool 210 is not limited to being closed on the side of the tool illustrated in FIG. 5. In some examples, handle 220 may include an opening on the side of insertion tool 210 shown in FIG. 5 so that a surface of the IMD received within the channel is visible from the perspective of insertion tool 210 shown in FIG. 5.

Handle 220 may include finger grips 226, 227. As seen in FIG. 5, finger grips 226, 227 may extend outwards from a longitudinal axis of insertion tool 210. Finger grips 226, 227 may be configured to allow a user to hold insertion tool 210 while using plunger 250. For example, it may require only one hand to hold insertion tool 210 and move plunger 250 distally within the channel formed by handle 220.

In the example of FIG. 5, a width of the blunt member 230 is approximately the same as a width of the sharp member 240. This means that the sharp member 240 obscures a large portion of the blunt member 230 from the perspective of FIG. 5. The width of the blunt member 230 is not limited to being approximately the same as a width of the sharp member 240. In some examples, the width of the blunt member 230 may be greater than width of the sharp member 240. Blunt member 230 may be configured to move along a longitudinal axis of the insertion tool 210 relative to sharp member 240. In the first configuration of insertion tool 210, the proximal end 242 of the sharp member 240 is close to the proximal end 222 of the handle 220. In some examples, the first configuration of insertion tool 210 shown in FIG. 5 represents the farthest extent to which the sharp member 240 can extend distally along the longitudinal axis of the insertion tool 210 relative to the blunt member 230. This means that the distal end 244 of sharp member 240 is distal to a distal end of blunt member 230.

Sharp member 240 may, in some cases, be restricted to movements relative to handle 220 and blunt member 230 along the longitudinal axis of insertion tool 210.

Handle 220, blunt member 230, and sharp member 240 may fit together such that sharp member 240 slides relative to handle 220 and blunt member 230. For example, sharp member 240 may include one or more crimped retentions that wrap around blunt member, the crimped retentions allowing the sharp member 240 to slide proximally and distally relative to blunt member 230, but preventing blunt member 230 from separating from sharp member 240. In some examples, blunt member 230 may include one or more crimped retentions that wrap around sharp member 240.

Plunger 250 is configured to move distally within a channel formed by handle 220. Plunger 250 includes a finger grip 256. The finger grip 256 is configured to allow a user to push the plunger distally within a channel formed by handle 220. A distal end 257 of the finger grip 256 may, in some cases, be wider than a portion of the plunger 250 distal to the distal end of the finger grip 256. This may prevent the plunger 250 from extending any further into the channel when the finger grip 256 reaches the handle 220.

Insertion tool 210 may be configured for sharp dissection when insertion tool 210 occupies the first configuration shown in FIG. 5. That is, when the distal end 244 of sharp member 240 is distal to the distal end of blunt member 230, the sharp distal portion of the sharp member 240 may be configured to pierce the skin of the patient to create an opening. When the distal end of the insertion tool 210 contacts skin while the insertion tool 210 is in the first position shown in FIG. 5, the sharp distal portion of sharp member 240 may pierce the skin by cutting the surface of the skin and advancing into the skin to create the opening. The blunt distal portion of the blunt member 230 is proximal to the sharp distal portion of the sharp member 240 when insertion tool 210 occupies the position shown in FIG. 5, meaning that the sharp distal portion of the sharp member 240 represents the distal portion of the insertion tool 210. This may allow the sharp distal portion of the sharp member 240 to pierce skin without being prevented from piercing or cutting the skin by blunt member 230.

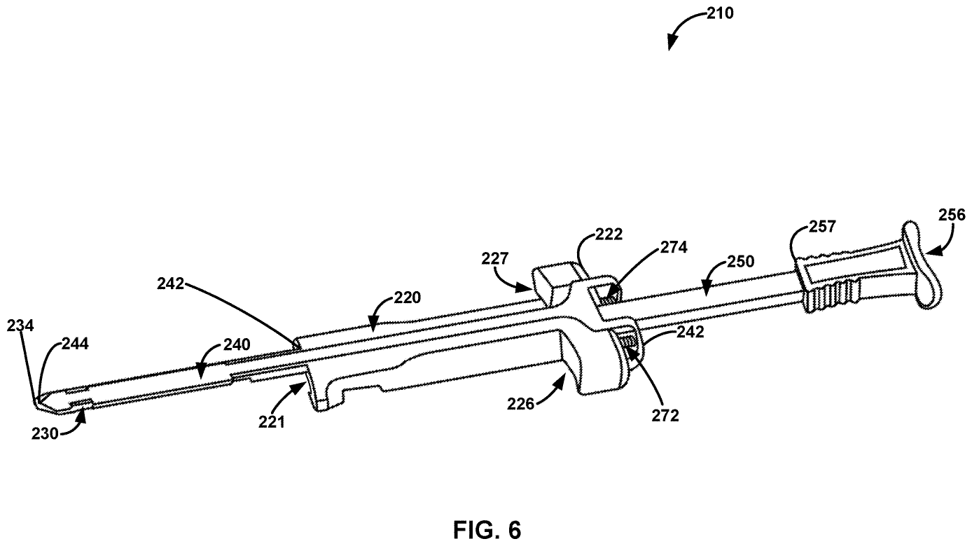

FIG. 6 is a conceptual diagram illustrating a perspective view of an insertion tool 210 for inserting an IMD in a patient while the insertion tool 210 occupies a second configuration, in accordance with one or more techniques described herein. That is, FIG. 6 may illustrate a different configuration of the insertion tool 210 illustrated by FIG. 5. As seen in FIG. 5, insertion tool 210 includes a handle 220, a blunt member 230, a sharp member 240, and a plunger 250. In some examples, handle 220 is an example of handle 20 of FIGS. 1A-2B, blunt member 230 is an example of blunt member 30 of FIGS. 1A-2B, sharp member 240 is an example of sharp member 40 of FIGS. 1A-2B, and plunger 250 is an example of plunger 50 of FIGS. 1A-2B.

The second configuration of insertion tool 210 represents a configuration where a distal portion of sharp member 240 is proximal to a distal portion of blunt member 230. For example, as seen in FIG. 6, the distal end 234 of blunt member 230 is distal to the distal end 244 of the sharp member 240. The distal portion of the blunt member 230 may include one or more blunt edges. The one or more blunt edges may, in some cases, be configured to separate subcutaneous tissue via blunt dissection without cutting the subcutaneous tissue. The distal portion of blunt member 230 might not be capable of piercing skin to create an opening.

Insertion tool 210 may be configured for blunt dissection when insertion tool 210 occupies the second configuration shown in FIG. 6. That is, when the distal end 244 of blunt member 230 is distal to the distal end of sharp member 240, the blunt distal portion of the blunt member 230 may be configured to create a pocket in subcutaneous tissue via blunt dissection. When the distal end of the insertion tool 210 enters an opening in the skin while the insertion tool 210 is in the second position shown in FIG. 6, the blunt distal portion of blunt member 230 may separate the subcutaneous tissue to create a pocket by advancing into the subcutaneous tissue. The blunt distal portion of the blunt member 230 is distal to the sharp distal portion of the sharp member 240 when insertion tool 210 occupies the position shown in FIG. 6, meaning that the blunt distal portion of the blunt member 230 represents the distal portion of the insertion tool 210. This may allow the blunt distal portion of the blunt member 230 to create the pocket via blunt dissection without sharp member 240 piercing or cutting the subcutaneous tissue.

Spring 272 and spring 274 (collectively, “springs 272, 274”) may, in some cases, be connected to both handle 220 and sharp member 240. Springs 272, 274 may be configured to extend and retract. For example, springs 272, 274 may extend as sharp member 240 transitions between the position illustrated by FIG. 5 and the position illustrated by FIG. 6, and springs 272, 274 may extend as sharp member 240 transitions between the position illustrated by FIG. 6 and the position illustrated by FIG. 5. When sharp member 240 is unrestrained from moving relative to handle 220 and blunt member 230, springs 272, 274 may extend and cause sharp member 240 to move proximally until sharp member 240 occupies the position illustrated by FIG. 6. One or more parts of insertion tool 210 may be configured to prevent sharp member 240 from moving. For example, a user control system may be configured to secure sharp member 240 in the position illustrated in FIG. 5 even when springs 272, 274 apply pressure to move sharp member 240 proximally. The user control system may include a user control (e.g., a button, a release mechanism) that is configured to secure sharp member 240 in the position illustrated by FIG. 5, and release sharp member 240 from the position illustrated by FIG. 5 in response to a user input to the user control. When the user control releases sharp member 240 from the position illustrated by FIG. 5, springs 272, 274 may cause sharp member 240 to move to the position illustrated by FIG. 6.

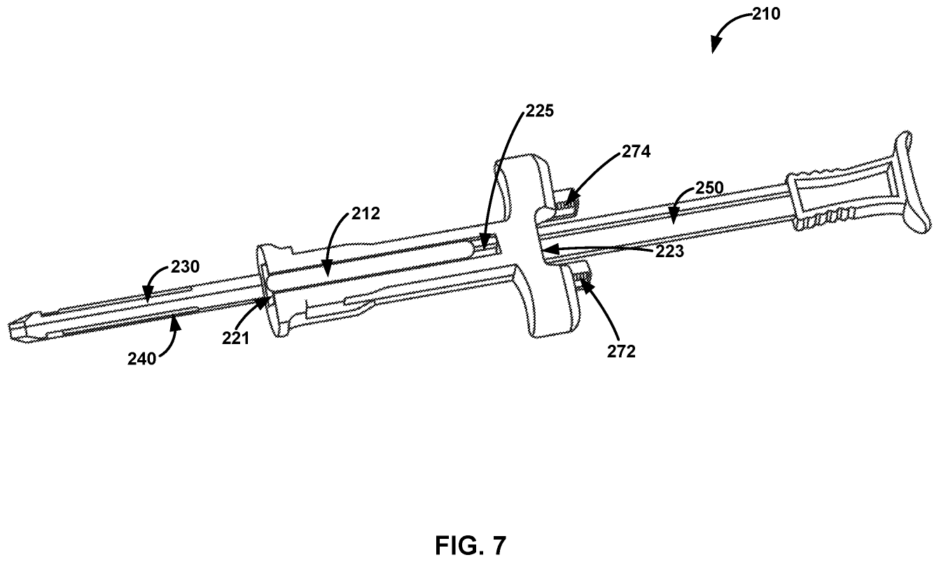

FIG. 7 is a conceptual diagram illustrating a perspective view of an insertion tool 210 including an IMD 212 within a channel formed by the insertion tool 210, in accordance with one or more techniques described herein. As seen in FIG. 7, insertion tool 210 includes a handle 220, a blunt member 230, a sharp member 240, and a plunger 250. In some examples, handle 220 is an example of handle 20 of FIGS. 1A-2B, blunt member 230 is an example of blunt member 30 of FIGS. 1A-2B, sharp member 240 is an example of sharp member 40 of FIGS. 1A-2B, and plunger 250 is an example of plunger 50 of FIGS. 1A-2B.

A channel formed by handle 220 of insertion tool 210 may receive IMD 212. In some examples, the perspective of FIG. 7 may include a side of the insertion tool 210 that is opposite a side of the insertion tool 210 visible in FIGS. 5 and 6. Handle 220 forms the channel such that the channel includes a distal opening 221 at a distal end of the handle 220, a proximal opening 223 at a proximal end of the handle 220, and a lateral opening 225 on a lateral surface of the handle 220. The IMD 212 and a distal portion of the plunger 250 may be visible within the channel through the lateral opening. When plunger 250 advances distally along a longitudinal axis of the insertion tool 210, plunger 250 may push IMD 212 out of the distal opening 221 of the channel formed by handle 220.

Insertion tool 210 as shown by FIG. 7 may occupy the same position as the insertion tool 210 shown in FIG. 6. Springs 272, 274 may cause sharp member 240 to occupy the same position as the insertion tool 210 shown in FIG. 6. This means that a distal end of the sharp member 240 is proximal to a distal end of the blunt member 230. A distal portion of sharp member 240 is obscured by the blunt member 230 from the perspective of FIG. 7. One or more crimped retentions of sharp member 240 that wrap around blunt member 230 are visible in FIG. 7.

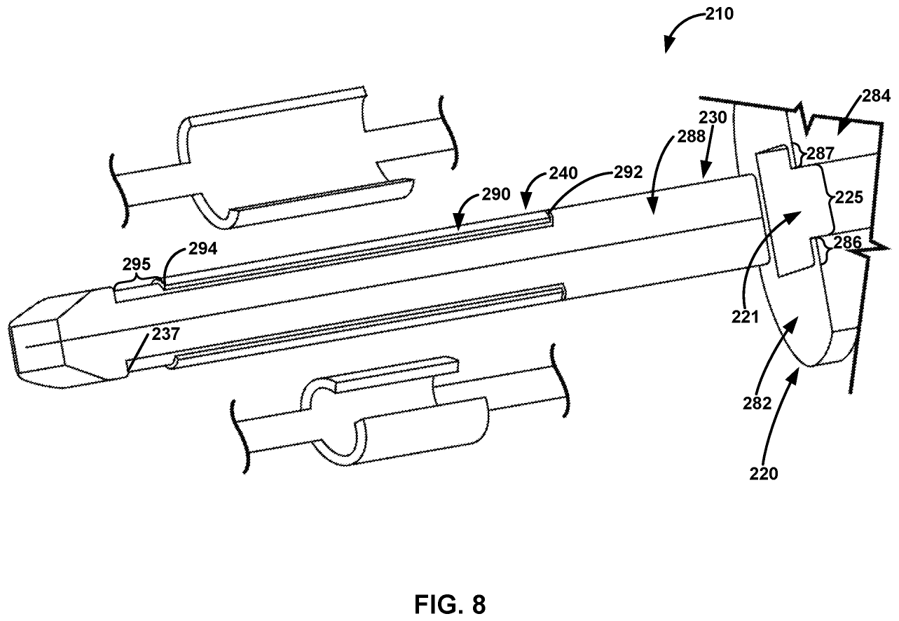

FIG. 8 is a conceptual diagram illustrating a perspective view of a distal portion of insertion tool 210, in accordance with one or more techniques described herein. As seen in FIG. 8, the distal portion of insertion tool 210 includes a distal portion of handle 220, blunt member 230, and a distal portion of sharp member 240.

Handle 220 includes a distal surface 282 and a lateral surface 284. In some examples, handle 220 may include a proximal surface opposite the distal surface 282 and a medial surface opposite the lateral surface 284. Handle 220 may form a channel that includes a distal opening 221 on the distal surface 282, and includes a lateral opening 225 on the lateral surface 284. In some examples, insertion tool 210 may receive a medical device, e.g., IMD 212, within the channel. IMD 212 may be configured to move along a longitudinal axis of insertion tool 210 both within the channel and outside of the channel. For example, a plunger, e.g., plunger 250, may push IMD 212 out of the distal opening 221 and onto a lateral surface 288 of blunt member 230.

When the medical device is received within the channel formed by handle 220, the medical device may be restricted to moving only along the longitudinal axis of the insertion tool 210. For example, although the medical device may be visible through lateral opening 225, lateral surface channel overhang 286 and lateral surface channel overhang 287 may prevent the medical device from moving laterally out of the channel. The medical device may be configured to exit the channel via the distal opening 221.

Sharp member 240 may include a crimped retention 290 that wraps around a portion of blunt member 230. Crimped retention 290 may extend from a proximal end 292 to a distal end 294. Crimped retention 290 may secure sharp member 240 to blunt member 230 and allow sharp member 240 to move along a longitudinal axis of insertion tool 210. However, movement of sharp member 240 along the longitudinal axis of insertion tool 210 may be restricted. For example, sharp member 240 may move proximally relative to blunt member 230 until the distal end 294 of crimped retention 290 reaches point 237. Blunt member 230 may include a surface at point 237 that prevents further proximal movement of sharp member 240. This means that sharp member 240 may be configured to move the distance of gap 295 between the distal end 294 of crimped retention 290 and point 237. When sharp member 240 moves proximally so that the distal end 294 of crimped retention 290 reaches point 237, a distal end of sharp member 240 may be distal to a distal end of blunt member 230.

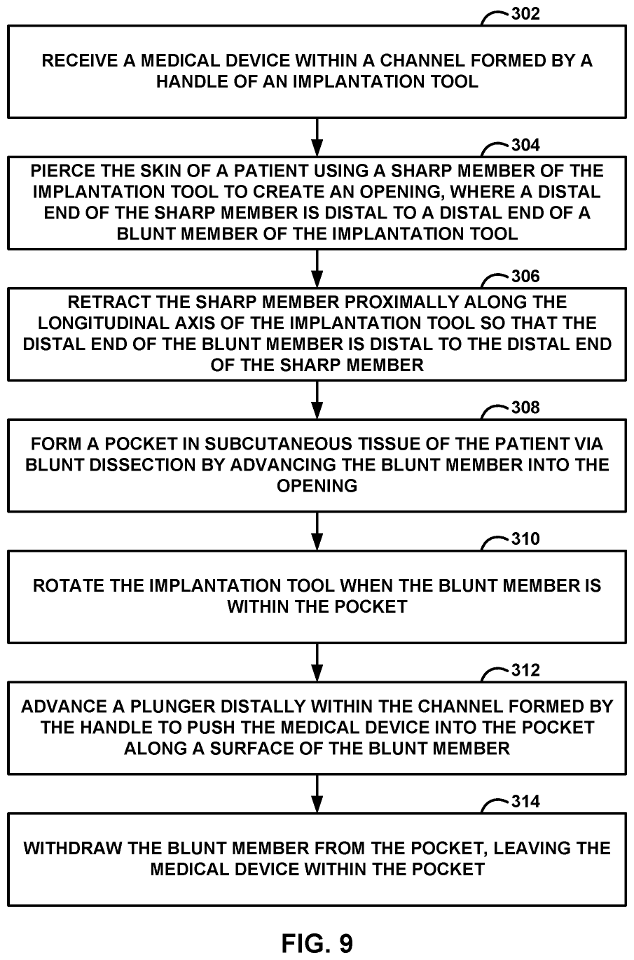

FIG. 9 is a flow diagram illustrating an example method for using an insertion tool to insert an IMD, in accordance with one or more techniques of this disclosure. FIG. 9 is described with respect to insertion tool 10 and IMD 12 of FIGS. 1-2. However, the techniques of FIG. 9 may be performed by different components of insertion tool 10 and IMD 12 or by additional or alternative medical devices or insertion tools.

Insertion tool 10 may receive an IMD 12 within a channel formed by a handle 20 of the insertion tool 10 (302). In some examples, handle 20 may form the channel so that the channel includes a distal opening on a distal surface of the handle 20, a proximal opening on a proximal surface of the handle 20, a lateral opening of a lateral surface of the handle 20, or any combination thereof. Handle 20 may include a medial surface opposite the lateral surface. In some examples, the medial surface includes a medial opening. In some examples, a blunt member 30 extends distally from handle 20. A sharp member 40 may be attached to handle 20 and/or blunt member 30 such that sharp member 40 is configured to move along longitudinal axis 14 relative to handle 20 and blunt member 30. In some examples, the proximal opening of the channel may receive a plunger, e.g., plunger 250, so that the plunger is configured to move within the channel. IMD 12 may exit the channel via the distal opening.

Insertion tool 10 may pierce the skin of a patient using sharp member 40 to create an opening, where a distal end 44 of sharp member 40 is distal to a distal end 34 of blunt member 30 (304). That is, sharp member 40 may pierce the skin of the patient to create the opening when the insertion tool 10 occupies the first position relative to handle 20 and blunt member 30 illustrated by FIGS. 1A and 2A. In some examples, sharp member 40 may pierce the skin of the patient to create the opening when a lateral surface of the insertion tool 10 faces upwards and away from the patient and when a medial surface of the insertion tool 10 faces downwards and towards the patient. This means that a caregiver may view the insertion tool 10 from the perspective shown in FIG. 2A when sharp member 40 pierces the skin of the patient to create the opening.

The sharp member 40 may retract proximally along the longitudinal axis 14 of insertion tool 10 so that the distal end 34 of the blunt member 30 is distal to a distal end 44 of sharp member 40 (306). In other words, sharp member 40 may transition from a first position first position relative to handle 20 and blunt member 30 as illustrated by FIGS. 1A and 2A to a second position relative to handle 20 and blunt member 30 as illustrated by FIGS. 1B and 2B. In some examples, the insertion tool 10 may include one or more springs between the proximal end 42 of the sharp member 40 and the proximal end 22 of the handle 20. The insertion tool 10 may include a user control system configured to secure the proximal end 42 of the sharp member 40 to the proximal end 22 of the handle 20 when the user control system is engaged. That is, the user control system may secure the sharp member 40 in the first position first position as illustrated by FIGS. 1A and 2A when the user control system is engaged. The user control system may allow the one or more springs to push the proximal end 42 of the sharp member 40 away from the proximal end 22 of the handle 20 when the user control system is released. That is, the user control system may allow the one or more springs to push the sharp member 40 to the second position as illustrated by FIGS. 1B and 2B.

Blunt member 30 may form a pocket in subcutaneous tissue of the patient via blunt dissection by advancing blunt member 30 into the opening (308). When the distal end 34 of blunt member 30 is distal to the distal end 44 of the sharp member 40, it may be possible to advance blunt member 30 into subcutaneous tissue and perform blunt dissection without cutting the tissue using sharp member 40. As seen in FIGS. 1B and 2B, the sharp distal portion of sharp member 40 that is configured for sharp dissection may be retreated such that the sharp distal portion does not engage with tissue as blunt member 30 advances into subcutaneous tissue.

The insertion tool 10 may rotate about the longitudinal axis 14 when the blunt member 30 is within the pocket (310). In other words, insertion tool 10 may transition from the lateral surface illustrated by FIGS. 2A and 2B facing upwards and away from the patient to the medial surface illustrated by FIGS. 1A and 1B facing upwards and away from the patient. Insertion tool 10 is configured to rotate 180 degrees about the longitudinal axis 14. Plunger 50 may advance distally within the channel formed by the handle 20 to push the IMD 12 into the pocket along a surface of blunt member 30 (312). In other words, the plunger 50 may advance the IMD 12 out of a distal opening of the channel and into the pocket while at least a portion of the blunt member 30 is inside of the pocket and the handle 20 is outside of the pocket. The blunt member 30 may withdraw from the pocket, leaving the IMD 12 within the pocket (314).

The following numbered clauses may demonstrate one or more aspects of the disclosure.

Clause 1: An insertion tool configured to insert a medical device in subcutaneous tissue of a patient, wherein the insertion tool comprises: a sharp member configured to pierce the subcutaneous tissue of the patient to create an opening, and a blunt member configured to form a pocket in the subcutaneous tissue via blunt dissection by advancing into the opening, wherein the sharp member is configured to move along a longitudinal axis of the insertion tool relative to the blunt member. The insertion tool also comprises a handle that forms a channel, wherein the handle is configured to receive the medical device in the channel; and a plunger configured to move in the channel, wherein the plunger is configured to push the medical device into the pocket as the plunger distally advances along the channel.

Clause 2: The insertion tool of clause 1, wherein the sharp member comprises a distal end and a proximal end, wherein the distal end of the sharp member is sharp so that the distal end of the sharp member is configured to pierce the subcutaneous tissue, wherein the blunt member comprises a distal end and a proximal end, wherein the distal end of the blunt member is blunt so that the distal end of the blunt member is configured to form the pocket in the subcutaneous tissue via blunt dissection, and wherein the handle comprises a distal end and a proximal end, wherein the blunt member extends distally from the distal end of the handle.

Clause 3: The insertion tool of clause 2, wherein as the proximal end of the sharp member advances distally along the longitudinal axis of the insertion tool towards the proximal end of the handle, the distal end of the sharp member advances distally along the longitudinal axis of the insertion tool so that the distal end of the sharp member is distal to a distal end of the blunt member.

Clause 4: The insertion tool of clause 3, wherein the distal end of the sharp member is configured to pierce the subcutaneous tissue when the distal end of the sharp member is distal to a distal end of the blunt member.

Clause 5: The insertion tool of any of clauses 2-4, wherein as the proximal end of the sharp member advances proximally along the longitudinal axis of the insertion tool away from the proximal end of the handle, the distal end of the sharp member advances proximally along the longitudinal axis of the insertion tool so that the distal end of the blunt member is distal to the distal end of the sharp member.

Clause 6: The insertion tool of clause 5, wherein the distal end of the blunt member is configured to form the pocket in the subcutaneous tissue via blunt dissection when the distal end of the blunt member is distal to the distal end of the sharp member.

Clause 7: The insertion tool of any of clauses 2-6, wherein the insertion tool comprises: one or more springs between the proximal end of the sharp member and the proximal end of the handle; and a user control system configured to: secure the proximal end of the sharp member to the proximal end of the handle when the user control system is engaged; and allow the one or more springs to push the proximal end of the sharp member away from the proximal end of the handle when the user control system is released.

Clause 8: The insertion tool of clause 7, wherein when the user control system is engaged, the user control system prevents the one or more springs from pushing the proximal end of the sharp member away from the proximal end of the handle, maintaining the distal end of the sharp member in a position distal to the distal end of the blunt member.

Clause 9: The insertion tool of any of clauses 7-8, wherein when the user control system is released, the one or more springs may push the proximal end of the sharp member away from the proximal end of the handle, maintaining the distal end of the sharp member in a position proximal to the distal end of the blunt member.

Clause 10: The insertion tool of any of clauses 1-9, wherein the channel is a first channel, wherein the blunt member and the handle are configured to form a second channel configured to receive the sharp member, and wherein the sharp member is configured to move along the longitudinal axis of the insertion tool within the second channel relative to the blunt member and the handle.

Clause 11: The insertion tool of any of clauses 1-10, wherein a distal portion of the sharp member is tapered from a central portion of the sharp member to form a sharp point at a distal end of the sharp member, and wherein the central portion of the sharp member comprises a first width, wherein a distal end of the blunt member is tapered from a central portion of the blunt member to form a blunt edge at a distal end of the blunt member, and wherein the central portion of the blunt member comprises a second width.

Clause 12: The insertion tool of clause 11, wherein the first width is within a range from 90% of the second width to 100% of the second width.

Clause 13: The insertion tool of any of clauses 11-12, wherein the first width is less than 90% of the second width.

Clause 14: The insertion tool of any of clauses 11-13, wherein the first width is less than 50% of the second width.

Clause 15: The insertion tool of any of clauses 1-14, wherein when the sharp member pierces the subcutaneous tissue of the patient, the medical device is outward of the blunt member relative to the patient's body, wherein when the blunt member advances into the subcutaneous tissue, the medical device is outward of the blunt member relative to the patient's body, and wherein the insertion tool is configured to rotate about the longitudinal axis such that the medical device is inward of the blunt member relative to the patient's body.

Clause 16: The insertion tool of clause 15, wherein the insertion tool is configured to rotate 180 degrees about the longitudinal axis.

Clause 17: The insertion tool of any of clauses 15-16, wherein the plunger is configured to push the medical device into the pocket when the insertion tool is rotated such that the medical device is inward of the blunt member relative to the patient's body.

Clause 18: A medical device system comprising: a medical device; and an insertion tool configured to insert the medical device in subcutaneous tissue of a patient. The insertion tool comprises: a sharp member configured to pierce the subcutaneous tissue of the patient to create an opening; and a blunt member configured to form a pocket in the subcutaneous tissue via blunt dissection by advancing into the opening, wherein the sharp member is configured to move along a longitudinal axis of the insertion tool relative to the blunt member. The insertion tool also includes a handle that forms a channel, wherein the handle is configured to receive the medical device in the channel; and a plunger configured to move in the channel, wherein the plunger is configured to push the medical device into the pocket as the plunger distally advances along the channel.

Clause 19: The medical device system of claim 18, wherein the medical device is an insertable cardiac monitor, the insertable cardiac monitor comprising: a power source operatively coupled to processing circuitry; a memory operatively coupled to processing circuitry; a distal electrode operatively coupled to the processing circuitry; a proximal electrode operatively coupled to the processing circuitry; and a hermetically-sealed housing configured for subcutaneous implantation within the patient, wherein at least the power source, memory, and processing circuitry are within the hermetically-sealed case, and wherein the housing has a length, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm.

Clause 20: A method of using an insertion tool to insert a medical device in subcutaneous tissue of a patient, the method comprising: piercing, using a sharp member of the insertion tool configured to insert the medical device in the subcutaneous tissue of the patient, the subcutaneous tissue of the patient to create an opening; and forming, using a blunt member of the insertion tool, a pocket in the subcutaneous tissue via blunt dissection by advancing into the opening, wherein the sharp member is configured to move along a longitudinal axis of the insertion tool relative to the blunt member. The method also includes receiving, by a handle of the insertion tool that forms a channel, the medical device in the channel; and pushing, by a plunger of the insertion tool configured to move in the channel, the medical device into the pocket as the plunger distally advances along the channel.

The techniques described in this disclosure may be implemented, at least in part, in hardware, software, firmware, or any combination thereof. For example, various aspects of the techniques may be implemented within one or more microprocessors, digital signal processors (DSPs), application specific integrated circuits (ASICs), field-programmable gate arrays (FPGAs), or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as random access memories (RAM), dynamic random access memories (DRAM), static random access memories (SRAM), magnetic discs, optical discs, flash memories, or forms of electrically programmable memories (EPROM) or electrically erasable and programmable memories (EEPROM). The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1. An insertion tool configured to insert a medical device in subcutaneous tissue of a patient, wherein the insertion tool comprises:
a sharp member configured to pierce the subcutaneous tissue of the patient to create an opening;
a blunt member configured to form a pocket in the subcutaneous tissue via blunt dissection by advancing into the opening, wherein the sharp member is configured to move along a longitudinal axis of the insertion tool relative to the blunt member;
a handle that forms a channel, wherein the handle is configured to receive the medical device in the channel; and
a plunger configured to move in the channel, wherein the plunger is configured to push the medical device into the pocket as the plunger distally advances along the channel.

2. The insertion tool of claim 1,
wherein the sharp member comprises a distal end and a proximal end, wherein the distal end of the sharp member is sharp so that the distal end of the sharp member is configured to pierce the subcutaneous tissue,
wherein the blunt member comprises a distal end and a proximal end, wherein the distal end of the blunt member is blunt so that the distal end of the blunt member is configured to form the pocket in the subcutaneous tissue via blunt dissection, and
wherein the handle comprises a distal end and a proximal end, wherein the blunt member extends distally from the distal end of the handle.

3. The insertion tool of claim 2, wherein as the proximal end of the sharp member advances distally along the longitudinal axis of the insertion tool towards the proximal end of the handle, the distal end of the sharp member advances distally along the longitudinal axis of the insertion tool so that the distal end of the sharp member is distal to a distal end of the blunt member.

4. The insertion tool of claim 3, wherein the distal end of the sharp member is configured to pierce the subcutaneous tissue when the distal end of the sharp member is distal to a distal end of the blunt member.

5. The insertion tool of claim 2, wherein as the proximal end of the sharp member advances proximally along the longitudinal axis of the insertion tool away from the proximal end of the handle, the distal end of the sharp member advances proximally along the longitudinal axis of the insertion tool so that the distal end of the blunt member is distal to the distal end of the sharp member.

6. The insertion tool of claim 5, wherein the distal end of the blunt member is configured to form the pocket in the subcutaneous tissue via blunt dissection when the distal end of the blunt member is distal to the distal end of the sharp member.

7. The insertion tool of claim 2, wherein the insertion tool comprises:
one or more springs between the proximal end of the sharp member and the proximal end of the handle; and
a user control system configured to:
secure the proximal end of the sharp member to the proximal end of the handle when the user control system is engaged; and
allow the one or more springs to push the proximal end of the sharp member away from the proximal end of the handle when the user control system is released.

8. The insertion tool of claim 7, wherein when the user control system is engaged, the user control system prevents the one or more springs from pushing the proximal end of the sharp member away from the proximal end of the handle, maintaining the distal end of the sharp member in a position distal to the distal end of the blunt member.

9. The insertion tool of claim 7, wherein when the user control system is released, the one or more springs may push the proximal end of the sharp member away from the proximal end of the handle, maintaining the distal end of the sharp member in a position proximal to the distal end of the blunt member.

10. The insertion tool of claim 1, wherein the channel is a first channel, wherein the blunt member and the handle are configured to form a second channel configured to receive the sharp member, and wherein the sharp member is configured to move along the longitudinal axis of the insertion tool within the second channel relative to the blunt member and the handle.

11. The insertion tool of claim 1,
wherein a distal portion of the sharp member is tapered from a central portion of the sharp member to form a sharp point at a distal end of the sharp member, and wherein the central portion of the sharp member comprises a first width,
wherein a distal end of the blunt member is tapered from a central portion of the blunt member to form a blunt edge at a distal end of the blunt member, and wherein the central portion of the blunt member comprises a second width.

12. The insertion tool of claim 1,
wherein when the sharp member pierces the subcutaneous tissue of the patient, the medical device is outward of the blunt member relative to the patient's body,
wherein when the blunt member advances into the subcutaneous tissue, the medical device is outward of the blunt member relative to the patient's body, and
wherein the insertion tool is configured to rotate about the longitudinal axis such that the medical device is inward of the blunt member relative to the patient's body.

13. The insertion tool of claim 12, wherein the plunger is configured to push the medical device into the pocket when the insertion tool is rotated such that the medical device is inward of the blunt member relative to the patient's body.

14. A medical device system comprising:
a medical device; and
an insertion tool configured to insert the medical device in subcutaneous tissue of a patient, wherein the insertion tool comprises:
a sharp member configured to pierce the subcutaneous tissue of the patient to create an opening;
a blunt member configured to form a pocket in the subcutaneous tissue via blunt dissection by advancing into the opening, wherein the sharp member is configured to move along a longitudinal axis of the insertion tool relative to the blunt member;
a handle that forms a channel, wherein the handle is configured to receive the medical device in the channel; and
a plunger configured to move in the channel, wherein the plunger is configured to push the medical device into the pocket as the plunger distally advances along the channel.

15. A method of using an insertion tool to insert a medical device in subcutaneous tissue of a patient, the method comprising:
piercing, using a sharp member of the insertion tool configured to insert the medical device in the subcutaneous tissue of the patient, the subcutaneous tissue of the patient to create an opening;
forming, using a blunt member of the insertion tool, a pocket in the subcutaneous tissue via blunt dissection by advancing into the opening, wherein the sharp member is configured to move along a longitudinal axis of the insertion tool relative to the blunt member;
receiving, by a handle of the insertion tool that forms a channel, the medical device in the channel; and
pushing, by a plunger of the insertion tool configured to move in the channel, the medical device into the pocket as the plunger distally advances along the channel.

16. The insertion tool of claim 11, wherein the first width is within a range from 90% of the second width to 100% of the second width.

17. The insertion tool of claim 11, wherein the first width is less than 90% of the second width.

18. The insertion tool of claim 11, wherein the first width is less than 50% of the second width.

19. The insertion tool of claim 12, wherein the insertion tool is configured to rotate 180 degrees about the longitudinal axis.

20. The medical device system of claim 18, wherein the medical device is an insertable cardiac monitor, the insertable cardiac monitor comprising:
a power source operatively coupled to processing circuitry;
a memory operatively coupled to processing circuitry;
a distal electrode operatively coupled to the processing circuitry;
a proximal electrode operatively coupled to the processing circuitry; and
a hermetically-sealed housing configured for subcutaneous implantation within the patient, wherein at least the power source, memory, and processing circuitry are within the hermetically-sealed case, and
wherein the housing has a length, a width, and a depth,
wherein the length is greater than the width and width is greater than the depth,
wherein the length is within a range from 5 millimeters (mm) to 60 mm,
wherein the width is within a range from 5 mm to 15 mm, and
wherein the depth is within a range from 5 mm to 15 mm.
